(19) United States
(12) Patent Application Publication
(10) Pub. No.: US 2003/0120073 A1
(43) Pub. Date: Jun. 26, 2003

(54) Alpha-ketocarboxylic acid based inhibitors of phosphoryl tyrosine phosphatases

(76) Inventor: Christopher T. Seto, Barrington, RI (US)

Correspondence Address: ROPES & GRAY, ONE INTERNATIONAL PLACE, BOSTON, MA, 02110-2624, US

(21) Appl. No.: 10/117,699

(22) Filed: Apr. 5, 2002

Related U.S. Application Data
(60) Provisional application No. 60/286,740, filed on Apr. 25, 2001.

(51) Int. Cl. C07F 7/02; C07D 213/63; C07D 213/72; C07D 213/70

(52) U.S. Cl. 546/14; 546/335; 546/290; 546/304; 546/314; 552/7; 558/414; 562/430; 560/358; 556/437

(57) ABSTRACT

Protein tyrosine phosphatases (PTPs) are implicated in a number of disease processes, including autoimmune diseases, glucose intolerance, diabetes, obesity and certain types of proliferative diseases such as cancer and psoriasis. The present invention discloses novel α-ketocarboxylic acid inhibitors of PTPs, methods for synthesizing said inhibitors, and methods for using pharmaceutical compositions of the inhibitors to treat PTP-mediated disorders, diseases or dysfunctions. The compositions described herein can also be used as diagnostic and screening tools to elucidate complex signal transduction pathways involving PTPs or to identify PTP-mediated disease states.

### REFERENCE TO RELATED APPLICATIONS

This application claims priority to U.S. Provisional Patent Application No. 60/286,740 filed on Apr. 25, 2001, which is incorporated by reference herein.

### BACKGROUND OF THE INVENTION

Phosphorylation states of proteins are governed by the opposing actions of two enzyme families: protein tyrosine kinases (PTKs), which catalyze the formation of phosphotyrosyl residues in peptide and protein substrates, and protein tyrosine phosphatases (PTPs), which are responsible for dephosphorylation of phosphorylated tyrosine residues. PTPs and PTKs play a central role in regulating cell growth, differentiation and metabolism. The interplay between PTPs and PTKs makes tyrosine phosphorylation of proteins a reversible and dynamic process. Many PTKs and PTPs and their associated substrates are thought to be integrated within elaborate signal transducing networks whose defective or inappropriate action can participate in widespread disease conditions such as autoimmune diseases, glucose intolerance, diabetes, obesity and certain types of proliferative diseases such as cancer and psoriasis. For instance, by catalyzing the removal of phosphoryl groups from phosphotyrosyl residues, PTPs can act both as “on” and “off” switches in signal transduction. Thus, modulation of the activities of specific PTPs may have beneficial therapeutic and diagnostic effects.

### SUMMARY OF THE INVENTION

The present invention relates to compositions and methods directed to inhibiting PTPs, methods of treatment for PTP-mediated disorders and dysfunctions, and methods for preparing said compositions and pharmaceutical preparations thereof.

The present invention also discloses methods for inhibiting PTPs, which can be useful as biochemical, diagnostic or screening tools. The invention further discloses methods for the preparation and use of pharmaceutical compositions of PTP inhibitors for treatments of PTP-mediated disorders. PTP-mediated disorders include, but are not limited to, autoimmune diseases, endocrine dysfunctions including glucose intolerance and type II diabetes, allergic disorders, and proliferative diseases including cancer and psoriasis.

Furthermore, the present invention discloses methods for conducting pharmaceutical business comprising the steps of drug formulation, drug testing, distribution, and licensing of drugs and diagnostic kits.

### DETAILED DESCRIPTION OF THE INVENTION

### A. OVERVIEW

The present invention discloses compositions that are inhibitors of PTPs and methods directed towards treating PTP-mediated disorders. In particular, the present invention discloses α-ketocarboxylic acid-based inhibitors of PTPs. The PTP-mediated disorders contemplated for treatment by the present invention include autoimmune diseases, glucose intolerance, diabetes, obesity, and certain types of proliferative diseases such as cancer and psoriasis.

### B. DEFINITIONS

(i) General Terms

The term “as valence and stability permit” in reference to compounds disclosed herein refers to compounds that have in vitro or in vivo half-lives at room temperature of at least 12 hours, or at least 24 hours, and are preferably capable of being stored at 0° C. for a week without decomposing by more than about 10%.

The term “clathrate” refers to inclusion compounds in which the guest molecule is in a cage formed by the host molecule or by a lattice of host molecules.

The term “ED₅₀” means the dose of a drug that produces 50% of its maximum response or effect.

The terms “half-life” or “half-lives” refer to the time required for half of a quantity of a substance to be converted to another chemically distinct species in vitro or in vivo.

The term “leaving group” refers to an atom or functional group which is easily displaced by a nucleophile or is easily eliminated in any one of the substitution or elimination reactions commonly known in organic chemistry to one of ordinary skill in the art. In general, leaving groups have conjugate acid dissociation constants (pKa) of about 4.5. Preferred leaving groups are halogens, phosphates, sulfates, and alkyl or aryl sulfonates. In certain cases, hydroxyl groups may be converted into leaving groups by transforming them into acetates. Hydroxyl groups can also be converted into leaving groups by treatment with a strong acid. Reaction with phosphorus, nitrogen, or sulfur reagents such as phosphorus pentahalides, thionyl chloride, and diethyl azodicarboxylate can also convert hydroxyl groups into leaving groups.

The term “metabolic derivative” refers to a compound derived by one or more in vitro or in vivo enzymatic transformations on the parent compound, wherein the resulting derivative has an ED₅₀ value as a PTP inhibitor that is less than 1000×ED₅₀ value of the parent compound.

The term “preventing” as used herein refers to delaying the onset or reducing the onset probability of a disease incident in a population that might be at risk for the disease.

The term “prodrug” refers to any compound that is converted to a more pharmacologically active compound under physiological conditions (e.g. in vivo). A common method for making a prodrug is to select moieties that are hydrolyzed under physiological conditions to provide the desired biologically active drug.

The term “treating” refers to: preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and relieving one or more symptoms of the disease, disorder or condition, i.e., causing regression of the disease, disorder and/or condition.

(ii) Chemical Terms

The term “acylamino” is art-recognized and refers to a moiety that can be represented by the general formula:

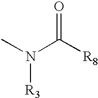

wherein R₃ is as defined below, and R₈ represents a hydrogen, an alkyl, an alkenyl or —(CH₂)ₘ-R₁, where m and R₁ are as defined below.

As used herein, the term “aliphatic group” refers to a straight chain, branched-chain, or cyclic aliphatic hydrocarbon group and includes saturated and unsaturated aliphatic groups, such as an alkyl group, an alkenyl group, and an alkynyl group.

The terms “alkenyl” and “alkynyl” refer to unsaturated aliphatic groups analogous in length and possible substitution to the alkyls described below, but that contain at least one double or triple bond respectively.

The terms “alkoxyl” or “alkoxy” as used herein refers to an alkyl group, as defined below, having an oxygen radical attached thereto. Representative alkoxyl groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O-(CH₂)ₘ-R₁, where m and R₁ are described below.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁—C₃₀ for straight chains, C₃—C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxyls, alkylthios, aminoalkyls, carbonyl-substituted alkyls, —CF₃, —CN, and the like.

Unless the number of carbons is otherwise specified, “lower alkyl”, as used herein, means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl. The term “alkylthio” refers to an alkyl group, as defined above, having a sulfur radical attached thereto. In preferred embodiments, the “alkylthio” moiety is represented by one of —(S)-alkyl, —(S)-alkenyl, —(S)-alkynyl, and —(S)—(CH₂)ₘ-R₁, wherein m and R₁ are defined below. Representative alkylthio groups include methylthio, ethylthio, and the like.

The terms “amine” and “amino” are art-recognized and refer to both unsubstituted and substituted amines, e.g., a moiety that can be represented by the general formulae:

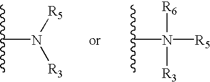

wherein R₃, R₅ and R₆ each independently represent a hydrogen, an alkyl, an alkenyl, —(CH₂)ₘ—R₁, or R₃ and R₅ taken together with the N atom to which they are attached complete a heterocycle having from 4 to 8 atoms in the ring structure; R₁ represents an alkenyl, aryl, cycloalkyl, a cycloalkenyl, a heterocyclyl or a polycyclyl; and m is zero or an integer in the range of 1 to 8. In preferred embodiments, only one of R₃ or R₅ can be a carbonyl, e.g., R₃, R₅ and the nitrogen together do not form an imide. In even more preferred embodiments, R₃ and R₅ (and optionally R₆) each independently represent a hydrogen, an alkyl, an alkenyl, or —(CH₂)ₘ-R₁. Thus, the term “alkylamine” as used herein means an amine group, as defined above, having a substituted or unsubstituted alkyl attached thereto, i.e., at least one of R₃ and R₅ is an alkyl group. In certain embodiments, an amino group or an alkylamine is basic, meaning it has a pKₐ ≧7.00. The protonated forms of these functional groups have pKₐs relative to water above 7.00.

The term “amido” is art-recognized as an amino-substituted carbonyl and includes a moiety that can be represented by the general formula:

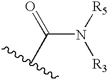

wherein R₃, R₅ are as defined above. Preferred embodiments of the amide will not include imides which may be unstable.

The term “aralkyl” or “arylalkyl”, as used herein, refers to an alkyl group substituted with an aryl group (e.g., an aromatic or heteroaromatic group).

The term “aryl” as used herein includes 5-, 6-, and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphate, phosphonate, phosphinate, carbonyl, carboxyl, silyl, sulfamoyl, sulfinyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused benzo rings”) wherein at least one of the rings is aromatic, e.g., the other cyclic rings can be cycloalkyls, cycloalkenyls, cycloalkynyls, aryls and/or heterocyclyls.

The term “carbocycle”, as used herein, refers to an aromatic or non-aromatic ring in which each atom of the ring is carbon. The carbocycle can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, or the like.

The term “carbonyl” is art-recognized and includes such moieties as can be represented by the general formula:

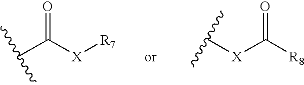

wherein X is a bond or represents an oxygen or a sulfur, and R₇ represents a hydrogen, an alkyl, an alkenyl, —(CH₂)ₘ-R₁ or a pharmaceutically acceptable salt, R₈ represents a hydrogen, an alkyl, an alkenyl or —(CH₂)ₘ-R₁, where m and R₁ are as defined above. Where X is an oxygen and R₇ or R₈ is not hydrogen, the formula represents an “ester”. Where X is an oxygen, and R₇ is as defined above, the moiety is referred to herein as a carboxyl group, and particularly when R₇ is a hydrogen, the formula represents a “carboxylic acid”. Where X is an oxygen, and R₈ is hydrogen, the formula represents a “formate”. In general, where the oxygen atom of the above formula is replaced by sulfur, the formula represents a “thiocarbonyl” group. Where X is a sulfur and R₇ or R₈ is not hydrogen, the formula represents a “thioester” group. Where X is a sulfur and R₇ is hydrogen, the formula represents a “thiocarboxylic acid” group. Where X is a sulfur and R₈ is hydrogen, the formula represents a “thioformate” group. On the other hand, where X is a bond, and R₇ is not hydrogen, the above formula represents a “ketone” group. Where X is a bond, and R₇ is hydrogen, the above formula represents an “aldehyde” group.

The term “heteroatom” as used herein means an atom of any element other than carbon or hydrogen. Preferred heteroatoms are boron, nitrogen, oxygen, phosphorus, sulfur and selenium.

The term “heavy atom” as used herein means includes any atom except hydrogen. Preferred heavy atoms are boron, carbon, nitrogen, oxygen, phosphorus, sulfur, and selenium.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, or the like.

As used herein, a “leaving group”

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, or the like.

The phrase “protecting group” as used herein means temporary substituents which protect a potentially reactive functional group from undesired chemical transformations. Examples of such protecting groups include esters of carbamates or amines, carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “sulfamoyl” is art-recognized and includes a moiety that can be represented by the general formula:

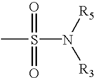

in which R₃ and R₅ are as defined above.

The term “sulfate” is art recognized and includes a moiety that can be represented by the general formula:

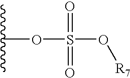

which R₇ is as defined above.

The term “sulfonamido” is art recognized and includes a moiety that can be represented by the general formula:

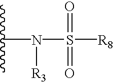

in which R₂ and R₄ are as defined above.

The term “sulfonate” is art-recognized and includes a moiety that can be represented by the general formula:

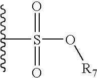

in which R₇ is an electron pair, hydrogen, alkyl, cycloalkyl, or aryl.

The terms “sulfoxido” or “sulfinyl”, as used herein, refers to a moiety that can be represented by the general formula:

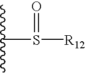

in which R₁₂ is selected from the group consisting of hydrogen, alkyl, alkenyl, alkynyl, cycloalkyl, heterocyclyl, aralkyl, or aryl.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g., alkyl, m, n, etc., when it occurs more than once in any structure, is intended to be independent of its definition elsewhere in the same structure.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

Certain compounds of the present invention may exist in particular geometric or stereoisomeric forms. The present invention contemplates all such compounds, including cis-and trans-isomers, (R)- and (S)-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof, wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

### C. EXEMPLARY EMBODIMENTS

In one aspect of the invention, the present invention discloses novel compositions of matter and their pharmaceutical compositions. One of ordinary skill in the art will recognize that esters and amide derivatives of the subject invention are within the ambit of the subject matter this of disclosure. Furthermore, the present invention encompasses metabolic derivatives of said compounds. These metabolic derivatives typically include, but are not limited to, hydroxylated aromatic derivatives of the compounds disclosed herein.

One embodiment of the invention is a compound, having the general structure shown in Formula I:

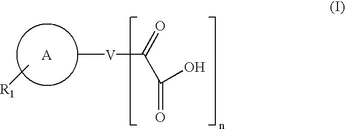

wherein, as valence and stability permits,

A represents an alkyl or heteroalkyl, or a substituted or unsubstituted aryl, heteroaryl, cycloalkyl, cycloalkenyl, heterocycloalkyl, or polycycle;

V, independently for each occurrence, is absent or represents a group selected from lower alkyl, alkoxy, arylalkyl, or a group with up to 10 heavy atoms;

R₁, independently for each occurrence, represents from one to six substituents on the moiety to which it is attached, selected from hydrogen, halogen, hydroxyl, alkoxyl, silyloxyl, amino, nitro, sulfhydryl, alkylthio, imine, amide, cyano, carbonyl, carboxyl, carboxamide, silyl, sulfamoyl, sulfinyl, thioalkyl, alkylsulfonyl, arylsulfonyl, ketone, aldehyde, ester, isocyano, guanidine, amidine, acetal, ketal, amine oxide, azide, carbamate, hydroxamic acid, imide, oxime, sulfonamide, thioamide, thiocarbonate, urea, thiourea, or a substituted or unsubstituted alkyl or heteroalkyl, alkenyl, alkynyl, aryl or heteroaryl or —(CH₂)ₘ-R₃;

R₃ represents a substituted or unsubstituted aryl, cycloalkyl, cycloalkenyl, heterocycloalkyl, heterocycle, or polycycle;

m, independently for each occurrence, represents an integer from 0 to 6; and

n is an integer from 1 to 4.

Another embodiment of the invention is a compound, having the general structure shown in Formula II:

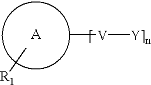

wherein, as valence and stability permits,

A represents an alkyl or heteroalkyl, or a substituted or unsubstituted aryl, heteroaryl, cycloalkyl, cycloalkenyl, heterocycloalkyl, or polycycle, preferably a substituted or unsubstituted phenyl, pyridinyl, thiazolyl, thienyl, furfuryl, oxazolyl, imidazolyl or pyrrolyl group;

Y, independently for each occurrence, represents,

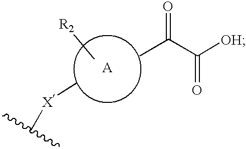

V, independently for each occurrence, is absent or represents a linking group selected from lower alkyl, alkenyl, alkynyl, alkoxy, or a group with up to 10 heavy atoms;

X′, independently for each occurrence, represents an O, NR₅or S preferably O;

R₁ and R₂, independently for each occurrence, represent from one to six substituents on the moiety to which it is attached, selected from hydrogen, halogen, hydroxyl, alkoxyl, silyloxyl, amino, nitro, sulfhydryl, alkylthio, imine, amide, cyano, carbonyl, carboxyl, carboxamide, silyl, sulfamoyl, sulfinyl, thioalkyl, alkylsulfonyl, arylsulfonyl, ketone, aldehyde, ester, isocyano, guanidine, amidine, acetal, ketal, amine oxide, azide, carbamate, hydroxamic acid, imide, oxime, sulfonamide, thioamide, thiocarbonate, urea, thiourea, or a substituted or unsubstituted alkyl, heteroalkyl, alkenyl, alkynyl, aryl or heteroaryl, or —(CH₂)ₘ-R₃; preferably selected from hydrogen, halogen, cyano, alkoxy, amino, nitro or carbonyl groups;

R₃ represents a substituted or unsubstituted aryl, cycloalkyl, cycloalkenyl, heterocycloalkyl, heterocycle, or polycycle;

R₅ represents a hydrogen or a group with up to 10 heavy atoms;

m, independently for each occurrence, represents an integer from 0 to 6, preferably from 1 to 3; and

n is an integer from 1 to 4; preferably from 2 to 3.

In certain embodiments, A represents a substituted or unsubstituted 5-6 fused aryl or heteroaryl ring.

In certain embodiments, A represents a substituted or unsubstituted cyclopropyl, cyclopentyl or cyclohexyl ring.

In certain embodiments, A is substituted by one or more halogen, lower alkyl, alkenyl, alkynyl, cyano, nitro, amino, or lower alkoxyl.

In certain embodiments A is a 6-6 fused aryl or heteroaryl ring.

Another aspect of the invention provides pharmaceutical preparations including a compound having the structure of Formula I or II, or a pharmaceutically acceptable salt, solvate, clathrate, pro-drug or metabolic derivative thereof.

In certain embodiments, the compounds of the invention described herein have a Kᵢ for inhibiting PTP activity in mammals, yeast, fungus or bacteria of less than or equal to 25 μM, 10 μM or even 1 μM.

In certain embodiments, the compounds of the invention described herein have an IC₅₀ for inhibiting PTP activity in mammals, yeast, fungus or bacteria of less than or equal to 10 μM or even 1 μM.

Another aspect of the invention is a pharmaceutical dosage form comprising a therapeutically effective amount of the compounds described herein. In certain embodiments, the dosage form is a tablet or a capsule or oral solution. In other embodiments, the dosage form is adapted for intravenous infusion, parenteral delivery or oral delivery. A therapeutically effective amount of the dosage form may be in the range of about 0.1 mg weight to about 1000 mg, or 1 mg to about 500 mg, 10 mg to about 100 mg, or even about 10 mg to about 75 mg.

Another aspect of the invention is a method for synthesizing a compound with a general structure shown in Formula III,

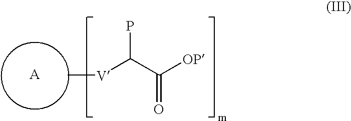

wherein, as valence and stability permits,

A represents an alkyl or heteroalkyl, or a substituted or unsubstituted aryl, heteroaryl, cycloalkyl, cycloalkenyl, heterocycloalkyl, or polycycle;

P represents a ketone protecting group or an optionally protected amine or hydroxyl;

P′ represents a protecting group for a carboxylic acid;

V′, independently for each occurrence, is absent or represents lower alkyl, alkenyl, alkynyl, alkoxy, or a group with up to 10 heavy atoms; and

m, independently for each occurrence, represents an integer from 1 to 4;

comprising,

reacting G with one or more equivalents of H in the presence of a base, wherein, as valence and stability permit,

G represents

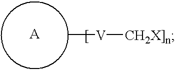

A represents alkyl or heteroalkyl, or a substituted or unsubstituted aryl, heteroaryl, cycloalkyl, cycloalkenyl, heterocycloalkyl, or polycycle;

V, independently for each occurrence, is absent or represents lower alkyl, alkenyl, alkynyl, alkoxy, or a group with up to 10 heavy atoms;

X is a leaving group preferably selected from halides, permethylated amines, acetates or sulphonate esters, such as a tosylate or mesylate; and n represents an integer in the range of 1 to 4;

H is represented by the general structure:

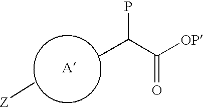

A′ represents an alkyl or heteroalkyl, or a substituted or unsubstituted aryl, heteroaryl, cycloalkyl, cycloalkenyl, heterocycloalkyl, or polycycle;

Z represents an OH, NR₅H, or SH;

R₅ represents a hydrogen or a group with up to 10 heavy atoms;

P′ represents a carboxyl protecting group; and

P represents a ketone protecting group or an optionally protected amine or hydroxyl.

Another aspect of the invention is a method for synthesizing a compound with a general structure shown in Formula IV,

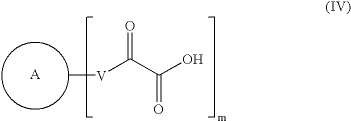

wherein, as valence and stability permits,

A represents an alkyl or heteroalkyl, or a substituted or unsubstituted aryl, heteroaryl, cycloalkyl, cycloalkenyl, heterocycloalkyl, or polycycle;

V, independently for each occurrence, is absent or represents a linking group selected from lower alkyl, alkenyl, alkynyl, alkoxy, or a group with up to 10 heavy atoms; and

m, independently for each occurrence, represents an integer from 1 to 4;

comprising the acts of

converting P to an oxo, and P′ to a hydrogen in the structure represented by formula IVa,

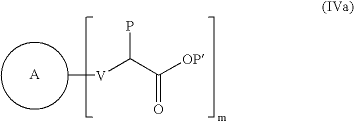

wherein, as valence and stability permits,

A represents an alkyl or heteroalkyl, or a substituted or unsubstituted aryl, heteroaryl, cycloalkyl, cycloalkenyl, heterocycloalkyl, or polycycle;

V, independently for each occurrence, is or represents a linking group selected from lower alkyl, alkenyl, alkynyl, alkoxy, or a group with up to 10 heavy atoms;

P represents a ketone protecting group or an optionally protected amine or hydroxyl;

P′ represents a protecting group for a carboxylic acid; and

m, independently for each occurrence, represents an integer from 1 to 4.

Another aspect of the invention is a method for synthesizing a compound with a general structure shown in Formula V,

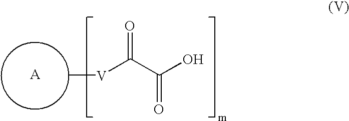

wherein, as valence and stability permits,

A represents an alkyl or heteroalkyl, or a substituted or unsubstituted aryl, heteroaryl, cycloalkyl, cycloalkenyl, heterocycloalkyl, or polycycle;

V, independently for each occurrence, is absent or represents a linking group selected from lower alkyl, alkenyl, alkynyl, alkoxy, or a group with up to 10 heavy atoms;

m, independently for each occurrence, represents an integer from 1 to 4;

comprising the act of converting P to an oxo in the compound represented by Formula Va:

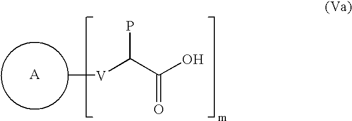

wherein, as valence and stability permits,

A represents an alkyl or heteroalkyl, or a substituted or unsubstituted aryl, heteroaryl, cycloalkyl, cycloalkenyl, heterocycloalkyl, or polycycle;

P represents a ketone protecting group;

V, independently for each occurrence, is absent or represents a linking group selected from lower alkyl, alkenyl, alkynyl, alkoxy, or a group with up to 10 heavy atoms; and

m, independently for each occurrence, represents an integer from 1 to 4.

Another aspect of the invention is a method for synthesizing a compound with a general structure shown in Formula VI,

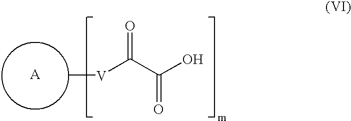

wherein, as valence and stability permits,

A represents an alkyl or heteroalkyl, or a substituted or unsubstituted aryl, heteroaryl, cycloalkyl, cycloalkenyl, heterocycloalkyl, or polycycle;

V, independently for each occurrence, is absent or represents a linking group selected from lower alkyl, alkenyl, alkynyl, alkoxy, or a group with up to 10 heavy atoms;

m, independently for each occurrence, represents an integer from 1 to 4;

comprising the act of converting P′ to a hydrogen in the compound represented by Formula VIa:

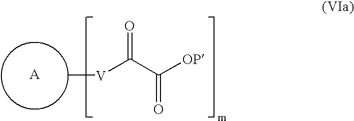

wherein, as valence and stability permits,

A represents an alkyl or heteroalkyl, or a substituted or unsubstituted aryl, heteroaryl, cycloalkyl, cycloalkenyl, heterocycloalkyl, or polycycle;

P′ represents a carboxy protecting group;

V, independently for each occurrence, is absent or represents a linking group selected from lower alkyl, alkenyl, alkynyl, alkoxy, or a group with up to 10 heavy atoms; and

m, independently for each occurrence, represents an integer from 1 to 4.

In certain embodiments, the removal of protecting groups (deprotection) may be accomplished in a substantially simultaneous fashion wherein the protecting groups are removed under the same reaction conditions. For example when P represents an acid-sensitive ketone protecting group such as ketal and P′ represents an acid-sensitive carboxyl protecting group such as a tert-butoxy group. Both acid-sensitive groups may be removed simultaneously by treatment with trifluoroacetic acid in dichloromethane. In certain embodiments, the deprotection steps may be achieved in a one-pot reaction sequence. In other embodiments, the deprotection step may be sequential wherein a P′ may be removed before P, or in the alternative P may be removed before P′. In sequential deprotections, two different reaction conditions may be used to remove P and P′, or there may be a purification process performed after the removal of P or P′. One of ordinary skill in the art will understand that the purification process may be a crude purification, such as an aqueous work-up followed by separation of the organic layer and evaporation of solvent, or an isolation of a purified chemical compound, e.g. using silica gel chromatography. Different reaction conditions may result from changes in reagents, temperature, pressure, or reaction times.

In certain embodiments P is a protected amine. In such cases, the conversion of P to an oxo involves first deprotecting the amine using one of a variety of techniques available to one of ordinary skill in the art. For instance, if the amine is protected by a tert-butyloxycarbonyl (Boc) group, one of ordinary skill in the art can remove the Boc group by treatment with a moderately acidic medium such as toluene sulfonic acid, or trifluoroacetic acid in dichloromethane or acetonitrile. The resulting amine may be converted to a carbonyl by a combination of transamination reaction and oxidative techniques, such as treatment with a hypervalent iodine oxidizing agent or a chromium (VI) salt.

In certain embodiments P is a protected hydroxyl. In such cases, the conversion of P to an oxo involves first deprotecting the hydroxyl using any of a variety of techniques available to one of ordinary skill in the art. For instance, if the hydroxyl is protected by an acid-sensitive protecting group, one of ordinary skill in the art can remove the protecting group by treatment with a mild acid medium such as pyridinium toluene sulphonate in tetrahydrofuran.

An exemplary synthetic scheme for synthesizing α-ketocarboxylic acid PTP inhibitors is presented in Scheme 1:

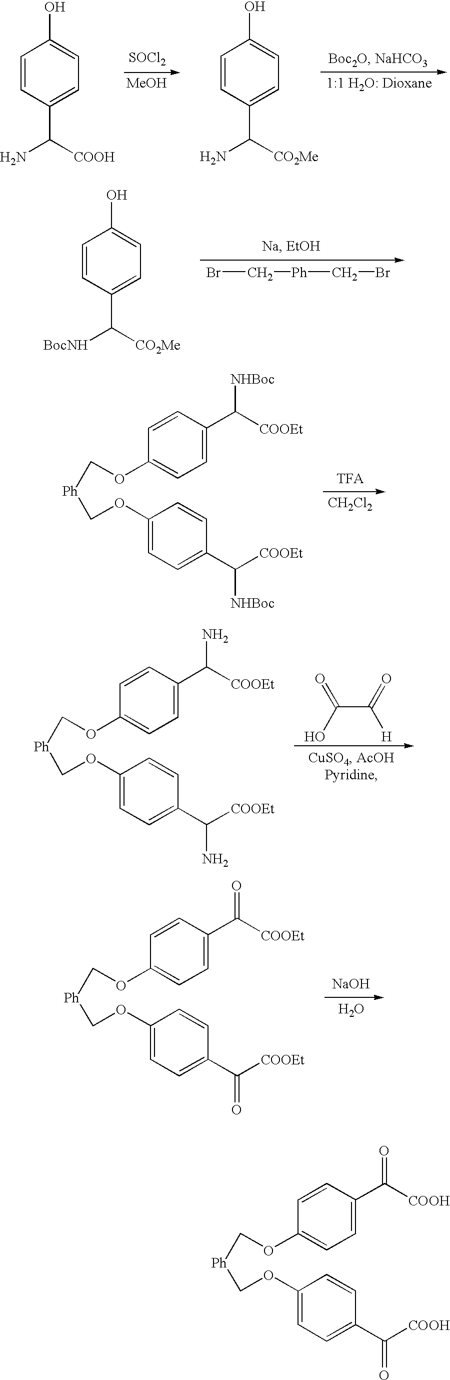

Another aspect of the invention is a method for inhibiting PTP activity in a subject comprising administering an effective amount of a pharmaceutical composition containing any of the compounds described herein.

Another aspect of the invention is a method for using the inhibitors disclosed herein as diagnostic or screening tools to elucidate PTP-mediated metabolic pathways and PTP-mediated disease states.

Another aspect of the invention is a method for treating a PTP-mediated disorder, the method comprising administering to a patient diagnosed with a PTP-mediated disorder a therapeutically effective amount of a pharmaceutical composition described herein. The PTP-mediated disorders may be selected from glucose intolerance, insulin resistance, type I diabetes, type II diabetes, obesity, autoimmune diseases, dysfunctions of the coagulation system, allergic diseases, diseases with decreased or increased synthesis or effects of growth hormones, osteoporosis, and proliferative disorders including cancer or psoriasis. In certain embodiments, the present invention encompasses a method of modulating glucose levels in a subject by administering a therapeutically effective amount of the pharmaceutical compositions described herein. The present invention further encompasses a method for treating metabolic disorders mediated by insulin resistance or hyperglycemia.

Another aspect of the present invention is a method for conducting a pharmaceutical business, comprising: (a) manufacturing a pharmaceutical composition containing any of the compounds described herein; and (b) marketing to healthcare providers the benefits of using the composition in the treatment of a PTP-mediated disorder, disease or dysfunction, in a patient, preferably a human, with instructions (written and/or pictorial) describing the use of the formulation for treatment or prevention of basal cell carcinoma, and, optionally, warnings of possible side effects and drug-drug or drug-food interactions.

In certain embodiments, the present invention is a method for conducting a pharmaceutical business, comprising: (a) providing a distribution network for selling a pharmaceutical composition containing any of the PTP inhibitors described herein; and (b) providing instruction material to patients or physicians for using the preparation in the treatment of a PTP-mediated disorder, disease or dysfimction, including instructions (written and/or pictorial) describing the use of the formulation for treatment or prevention of basal cell carcinoma, and, optionally, warnings of possible side effects and drug-drug or drug-food interactions.

In certain embodiments, the present invention is method for conducting a pharmaceutical business, comprising: (a) determining an appropriate formulation and dosage of a pharmaceutical composition containing any of the PTP inhibitors disclosed herein; (b) conducting therapeutic profiling of formulations identified in step (a) for efficacy and toxicity in animals; and (c) providing a distribution network for selling a preparation or preparations identified in step (b) as having an acceptable therapeutic profile.

In still further embodiments, the method includes an additional step of providing a sales group for marketing (e.g., providing promotional and/or informative presentations (such as displays, telemarketing, and lectures), products (such as trial samples of the preparation), and/or documentation (including leaflets, pamphlets, websites, posters, etc.)) to healthcare providers, such as doctors, hospitals, clinics, etc., a benefit of using the pharmaceutical preparation for treating or preventing PTP-mediated disorders.

In yet other embodiments, the invention provides a method for conducting a pharmaceutical business, comprising: (a) determining an appropriate formulation and dosage of a pharmaceutical composition described herein; and (b) licensing, to a third party, the rights for further development and sale of the formulation.

D. EXEMPLARY FORMULATIONS

In another aspect, the present invention provides pharmaceutical compositions. The composition for use in the subject method may be conveniently formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to one of ordinary skill in the arts. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the PTP inhibition, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Pharmaceutical formulations of the present invention can also include veterinary compositions, e.g., pharmaceutical preparations of the PTP inhibitors suitable for veterinary uses, e.g., for the treatment of livestock or domestic animals, e.g., dogs.

Administration may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a drug at a particular target site.

The preparations of the present invention may be given orally, parenterally, topically, or rectally. They are, of course, given by forms suitable for each administration route. For example, they are administered in tablets or capsule form, by injection, inhalation, eye lotion, ointment, suppository, controlled release patch, administration by injection, infusion or inhalation; topical by lotion or ointment; and rectal by suppositories. Oral and topical administrations are preferred.

The phrases “parenteral administration” or “administered parenterally” as used herein mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect, and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The phrase “therapeutically effective amount” as used herein means that amount of a compound, material, or composition comprising a compound of the present invention which is effective for producing some desired therapeutic effect by inhibiting PTPs in at least a sub-population of cells in an animal and thereby blocking the biological consequences of that pathway in the treated cells, at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its analogs, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present composition may contain a basic functional group, such as amino or alkylamino, and are thus, capable of forming pharmaceutically acceptable salts with pharmaceutically acceptable acids. The term “pharmaceutically acceptable salts” in this respect refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, α-tocopherol, and the like; and (3) metal chelating agents, such as citric acid, ethylenediamine tetraacetic acid (EDTA), sorbitol, tartaric acid, phosphoric acid, and the like.

Pharmacological dosages or formulations of the present invention include those suitable for oral, nasal, topical (including buccal and sublingual), rectal, vaginal and/or parenteral administration. The dosages may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth-washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active ingredient.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose analogs, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the composition in the proper medium. Absorption enhancers can also be used to increase the flux of the composition across the skin. The rate of such flux can be controlled by either providing a rate-controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W. H. Freedman and CO., San Francisco, U.S.A., 1969 or “Livestock Feeds and Feeding” O and B books, Corvallis, Oreg., U.S.A., 1977).

### E. EXAMPLES

i. Synthetic Scheme

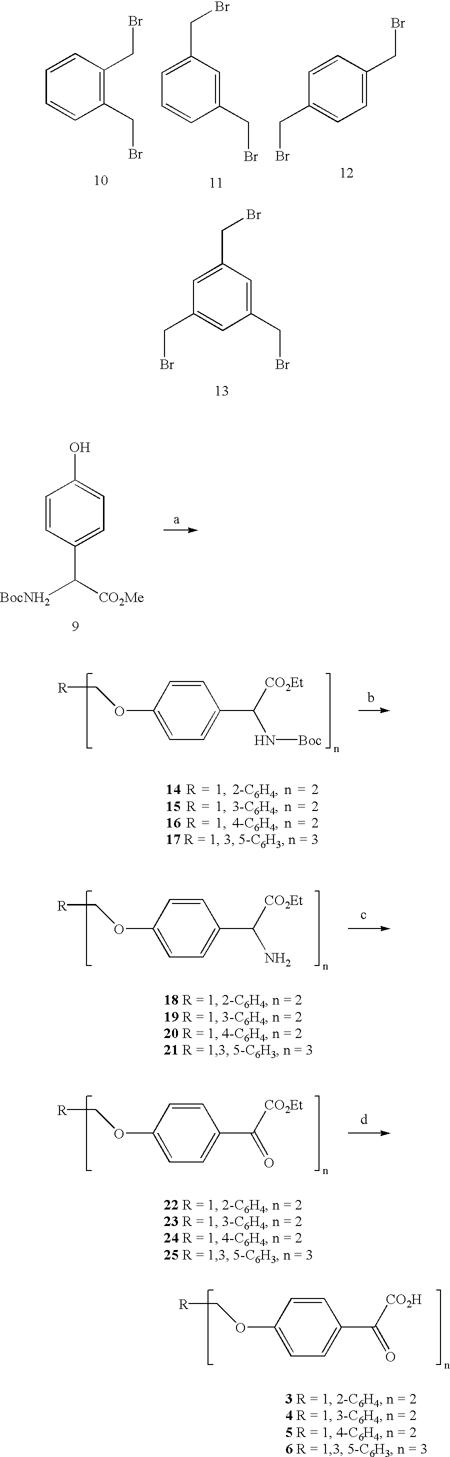

^{α}Reagents: (a) Na, EtOH; (b) TFA, CH₂Cl₂; (c) HO₂CCHO, CuSO₄, AcOH, pyridine, (d) NaOH, H₂O.

### Example 1

D-4-Hydroxyphenylglycine methyl ester: D-4-Hydroxyphenylglycine (7, 10.0 g, 60.1 mmol) was suspended in methanol (200 mL) and thionyl chloride (8 mL) was added dropwise. After the resulting mixture was stirred at room temperature for 10 h, the solvent was removed by rotary evaporation and the residue was washed twice with ether to yield D-4-hydroxyphenylglycine methyl ester as a white solid (13.0 g, 60.0 mmol, 100%): ¹H NMR (300 MHz, DMSO-d₆) δ3.68 (s, 3 H), 5.07 (s, 1 H), 6.85 (d, J=8.5 Hz, 2 H), 7.29 (d, J=8.6 Hz, 2 H), 9.03 (s, 3 H), 10.02 (s, 1 H); ¹³C NMR (75 MHz, DMSO-d₆) δ53.8, 55.8, 116.5, 123.4, 130.5, 159.4, 170.0; LRMS-FAB (M+H⁺) calcd for C₉H₁₂NO₃ 182, found 182.

### Example 2

N-Boc-D-4-Hydroxyphenylglycine methyl ester (9): To a mixture 8 (2.60 g, 12.0 mmol) and NaHCO₃ (1.51 mg, 18.0 mmol) in H₂O (25 mL) was added Boc anhydride (3.13 g, 14.3 mmol) in dioxane (25 mL). The mixture was stirred for 1 h at 0° C. then 6 h at room temperature. After acidifying with 1 N HCl to approximately pH 2, the mixture was extracted with three portions of ethyl acetate (5 mL). The organic layer was combined and washed with H₂O (10 mL) and brine (10 mL), dried over Na₂SO₄, and the solvent was removed by rotary evaporation to yield N-Boc-D-4-hydroxyphenylglycine methyl ester as a white solid (3.60 g, 11.3 mmol, 95%): ¹H NMR (300 MHz, CDCl₃) δ1.45 (s, 9 H), 3.71 (s, 3 H), 5.23 (d, J=7.1 Hz, 1 H), 5.61 (d, J=6.5 Hz, 1 H), 6.56 (s, 1 H), 6.75 (d, J=8.6 Hz, 2 H), 7.17 (d, J=8.1 Hz, 2 H); ¹³C NMR (75 MHz, CDCl₃) δ28.7, 53.1, 57.5, 80.9, 116.2, 128.6, 128.8, 147.2, 155.5, 172.4; LRMS-FAB (M +Na⁺) calcd for C₁₄H₁₉NNaO₅ 304, found 304.

### Example 3

1,3,5-α,α′,α″-tribromomesitylene: This compound was synthesized according to the procedure of Vogel, F.; Zuber, M.; Lichtenthaler, R. G. Chem. Ber. 1973, 106, 717-718.

### Example 4

General Procedure for the Synthesis of Compounds 14-17: Ether 15. To a solution of sodium (0.302 g, 13.1 mmol) dissolved in ethanol (50 mL) was added 9 (3.09 g, 11.0 mmol) and then α,α′-dibromo-m-xylene (11, 1.61 g, 4.40 mmol). The mixture was stirred continuously at room temperature for 23 h under a N₂ atmosphere, the solvent was removed, and the residue was treated with H₂O (30 mL) and ethyl acetate (30 mL). The layers were separated, and the aqueous layer was extracted with two portions of ethyl acetate (30 mL). The organic layers were combined and washed with H₂O (30 mL), twice with a saturated solution of aqueous Na₂CO₃ (30 mL), and brine (30 mL), dried over Na₂SO₄, and concentrated to dryness. Purification by flash column chromatography (1:1 hexane:ethyl ether) yielded compound 15 (1.49 g, 2.15 mmol, 49%) as white foam: ¹H NMR (300 MHz, CDCl₃) δ1.23 (t, J=7.1 Hz, 6 H), 1.45 (s, 18 H), 4.19 (m, 4 H), 5.08 (s, 4 H), 5.25 (d, J=7.3 Hz, 2 H), 5.53 (d, J=6.6 Hz, 2 H), 6.96 (d, J=8.7 Hz, 4 H), 7.30 (d, J=8.7 Hz, 4 H), 7.41 (s, 3 H), 7.50 (s, 1 H); ¹³C NMR (75 MHz, CDCl₃) δ14.4, 28.7, 57.5, 62.1, 70.2, 80.5, 115.5, 126.9, 127.5, 128.8, 129.3, 129.9, 137.6, 155.3, 159.1, 171.8; ESI (M+H⁺) calcd for C₃₈H₄₉N₂O₁₀ 693.3387, found 693.3369.

### Example 5

Ether 14: This compound was synthesized in 43% yield: ¹H NMR (300 MHz, CDCl₃) δ1.23 (t, J=7.1 Hz, 6 H), 1.45 (s, 18 H), 4.19 (m, 4 H), 5.16 (s, 4 H), 5.25 (d, J=7.9 Hz, 2 H), 5.52 (m, 2 H), 6.95 (d, J=8.4 Hz, 4 H), 7.29 (d, J=8.4 Hz, 4 H), 7.39 (dd, J=5.6, 3.3 Hz, 2 H), 7.52 (dd, J=5.5, 3.5 Hz, 2 H); ¹³C NMR (75 MHz, CDCl₃) δ14.4, 28.7, 57.4, 62.1, 68.4, 80.4, 115.5, 128.8, 129.0, 129.4, 130.1, 135.3, 155.2, 159.0, 171.7; ESI (M+H⁺) calcd for C₃₈H₄₉N₂O₁₀ 693.3387, found 693.3407.

### Example 6

Ether 16: This compound was synthesized in 47% yield: ¹H NMR (300 MHz, CDCl₃) δ1.23 (t, J=7.1 Hz, 6 H), 1.45 (s, 18 H), 4.19 (m, 4 H), 5.07 (s, 4 H), 5.25 (d, J=6.9 Hz, 2 H), 5.51 (d, J =6.6 Hz), 6.96 (d, J=8.7 Hz, 4 H), 7.30 (d, J=8.7 Hz, 4 H), 7.45 (s, 4 H); ¹³C NMR (75 MHz, CDCl₃) δ14.4, 28.7, 57.5, 62.1, 70.1, 80.4, 115.5, 128.1, 128.8, 130.0, 137.1, 155.2, 159.1, 171.7; ESI (M+H⁺) calcd for C₃₈H₄₉N₂O₁₀ 693.3387, found 693.3395.

### Example 7

Ether 17: This compound was synthesized in 21% yield: ¹H NMR (300 MHz, CDCl₃) δ1.23 (t, J=7.1 Hz, 9 H), 1.45 (s, 27 H), 4.20 (m, 6 H), 5.09 (s, 6 H), 5.26 (d, J=7.1 Hz, 3 H), 5.54 (d, J =6.0 Hz, 3 H), 6.96 (d, J=8.7 Hz, 6 H), 7.31 (d, J=8.7 Hz, 6 H) 7.46 (s, 3 H); ¹³C NMR (75 MHz, CDCl₃) δ14.4, 28.7, 57.5, 62.1, 70.1, 80.5, 115.5, 126.4, 128.8, 130.1, 138.2, 155.2, 159.1, 171.7; ESI (M+H⁺) calcd for C₅₄H₇₀N₃O₁₅ 1000.4807, found 1000.4817.

### Example 8

General Procedure for the Synthesis of Compounds 18-21: Amine 19. Compound 15 (1.85 g, 2.67 mmol) was dissolved in a solution of 20% TFA in methylene chloride (10 mL) and the solution was stirred in an ice bath for 2 h. The solvent was removed, and the residue was taken up in methylene chloride (30 mL) and washed twice with a saturated solution of aqueous NaHCO₃ (20 mL). The organic layer was dried over Na₂SO₄ and the solvent was removed by rotary evaporation to yield compound 19 (1.19 g, 2.42 mmol, 91%) as a clear oil: ¹H NMR (300 MHz, DMSO-d₆) δ1.11 (t, J=7.1 Hz, 6 H), 2.19 (s, 4 H), 4.04 (m, 4 H), 4.42 (s, 2 H), 5.09 (s, 4 H), 6.96 (d, J=8.5 Hz, 4 H), 7.28 (d, J=8.5 Hz, 4 H), 7.39 (s, 3 H), 7.51 (s, 1 H); ¹³C NMR (75 MHz, DMSO-d₆) δ14.9, 55.8, 58.4, 61.1, 69.9, 115.4, 127.7, 128.0, 129.4, 134.3, 138.2, 158.5, 175.1; ESI (M+H⁺) calcd for C₂₈H₃₃N₂O₆ 493.2339, found 493.2344.

### Example 9

Amine 18: This compound was synthesized in 85% yield: ¹H NMR (300 MHz, DMSO-d₆) δ1.12 (t, J=7.1 Hz, 6 H), 2.21 (s, 4 H), 4.04 (s, 4 H), 4.42 (s, 2 H), 5.21 (s, 4 H), 6.97 (d, J=8.7 Hz, 4 H), 7.28 (d, J=8.7 Hz, 4 H), 7.36 (dd, J=5.6, 3.4 Hz, 2 H), 7.51 (dd, J=5.4, 3.4 Hz, 2 H); ¹³C NMR (75 MHz, DMSO-d₆) δ14.9, 58.5, 61.1, 112.7, 115.4, 128.8, 129.3, 134.4, 136.0, 158.4, 175.0; ESI (M+H⁺) calcd for C₂₈H₃₃N₂O₆ 493.2339, found 493.2340.

### Example 10

Amine 20: This compound was synthesized in 86% yield: ¹H NMR (300 MHz, DMSO-d₆) δ1.12 (t, J=7.1 Hz, 6 H), 2.21 (s, 4 H), 4.04 (m, 4 H), 4.42 (s, 2 H), 5.09 (s, 4 H), 6.95 (d, J=8.7 Hz, 4 H), 7.28 (d, J=8.7 Hz, 4 H), 7.44 (s, 4 H); ¹³C NMR (75 MHz, DMSO-d₆) δ14.9, 58.4, 61.1, 69.8, 115.4, 128.6, 128.8, 134.2, 137.6, 158.4, 175.0; ESI (M+H⁺) calcd for C₂₈H₃₃N₂O₆ 493.2339, found 493.2327.

### Example 11

Amine 21: This compound was synthesized in 75% yield: ¹H NMR (300 MHz, DMSO-d₆) δ1.12 (t, J=7.1 Hz, 9 H), 2.17 (s, 6 H), 4.05 (m, 6 H), 4.43 (s, 3 H), 5.12 (s, 6 H), 6.97 (d,J=7.6 Hz, 6 H), 7.29 (d, J=7.8 Hz, 6 H), 7.48 (s, 3 H); ¹³C NMR (300 MHz, DMSO-d₆) δ14.9, 58.4, 61.1, 69.8, 115.4, 127.2, 128.8, 134.3, 138.5, 158.4, 175.1; ESI (M+H⁺) calcd for C₃₉H₄₆N₃O₉ 700.3234, found 700.3252.

### Example 12

General Procedure for the Synthesis of Compounds 22-25: (Ketoester 23) To a solution of 19 (0.344 g, 0.70 mmol) in dioxane (6 mL) was added a freshly prepared aqueous solution (10 mL) of glyoxylic acid (0.736 g, 8.0 mmol) and copper (II) sulfate pentahydrate (200 mg, 0.80 mmol) in a buffer containing 2.5 M pyridine and 0.5 M acetic acid. The mixture was stirred overnight at room temperature and then extracted with three portions of methylene chloride (25 mL). The organic layers were combined, washed twice with 1 N HCl (20 mL), dried over Na₂SO₄, and then concentrated to dryness. The residue was purified by flash column chromatography (CHCl₃) to yield 23 (66.4 mg, 0.14 mmol, 19%) as clear oil: ¹H NMR (300 MHz, CDCl₃) δ1.44 (t, J=7.1 Hz, 6 H), 4.45 (q, J=7.1 Hz, 4 H), 5.20 (s, 4 H), 7.07 (d, J=8.9 Hz, 4 H), 7.44 (m, 3 H), 7.52 (s, 1 H), 8.04 (d, J=8.9 Hz, 4 H); ¹³C NMR (75 MHz, CDCl₃) δ14.5, 62.6, 70.4, 115.4, 126.3, 126.8, 127.8, 129.6, 133.0, 136.9, 164.3, 164.5, 185.2; ESI (M+H⁺) calcd for C₂₈H₂₇O₈ 491.1706, found 491.1721.

### Example 13

Ketoester 22. This compound was synthesized in 16% yield: ¹H NMR (300 MHz, DMSO-d₆) δ1.31 (t, J=7.1 Hz, 6 H), 4.39 (q, J=7.1 Hz, 4 H), 5.39 (s, 4 H), 7.21 (d, J=8.9 Hz, 4 H), 7.42 (dd, J=5.5, 3.5 Hz, 2 H), 7.56 (dd, J=5.5, 3.5 Hz, 2 H), 7.91 (d, J=8.9 Hz, 4 H); ¹³C NMR (75 MHz, DMSO-d₆) δ14.7, 63.0, 68.6, 116.4, 125.6, 129.4, 129.8, 133.1, 135.3, 164.7, 165.0, 186.1; ESI (M+Na⁺) calcd for C₂₈H₂₆NaO₈ 513.1526, found 513.1502.

### Example 14

Ketoester 24. This compound was synthesized in 21% yield: ¹H NMR (300 MHz, CDCl₃) δ1.44 (t, J=7.1 Hz, 6 H), 4.45 (q, J=7.1 Hz, 4 H), 5.19 (s, 4 H), 7.06 (d, J=8.8 Hz, 4 H), 7.48 (s, 4 H), 8.03 (d, J=8.8 Hz, 4 H); ¹³C NMR (75 MHz, CDCl₃) δ14.5, 62.6, 70.3, 115.4, 126.2, 128.3, 133.0, 136.4, 164.4, 164.5, 185.2; ESI (M+Na⁺) calcd for C₂₈H₂₆NaO₈ 513.1526, found 513.1514.

### Example 15

Ketoester 25. This compound was synthesized in 15% yield: ¹H NMR (300 MHz, CDCl₃) δ1.44 (t, J=7.1 Hz, 9 H), 4.45 (q, J=7.1 Hz, 6 H), 5.21 (s, 6 H), 7.07 (d, J=8.9 Hz, 6 H), 7.50 (s, 3 H), 8.04 (d, J=8.9 Hz, 6 H); ¹³C NMR (75 MHz, CDCl₃) δ14.5, 62.6, 70.1, 115.4, 126.4, 126.6, 133.1, 137.6, 164.2, 164.4, 185.1; ESI (M+H⁺) calcd for C₃₉H₃₇O₁₂ 697.2285, found 697.2306.

### Example 16

General Procedure for the Synthesis of Compounds 3-6: (Ketoacid 4) To a solution of compound 23 (66.4 mg, 0.135 mmol) in methanol (2 mL) was added 2.5 M NaOH (2 mL). The mixture was stirred at 50° C. for 1 h, cooled, and acidified with concentrated HCl to approximately pH 2. The precipitate was collected by centrifugation, and resuspended in water (5 mL). Addition of ethyl acetate (5 mL) dissolved the precipitate, and after the layers were separated, the aqueous layer was extracted twice with ethyl acetate (5 mL). The ethyl acetate layers were combined, dried over Na₂SO₄, and concentrated to dryness to yield 4 as a yellow solid (40.5 mg, 0.093 mmol, 69%): ¹H NMR (300 MHz, DMSO-d₆) δ5.27 (s, 4 H), 7.22 (d, J=8.8 Hz, 4 H), 7.46 (s, 3 H), 7.59 (s, 1 H), 7.89 (d, J=8.8 Hz, 4 H); ¹³C NMR (75 MHz, DMSO-d₆) δ70.4, 116.3, 125.8, 128.2, 128.5, 129.7, 132.8, 137.4, 164.5, 167.4, 188.1; ESI (M−H⁺, negative ion mode) calcd for C₂₄H,₁₇O₈ 433.0923, found 433.0932.

### Example 17

Ketoacid 3. This compound was synthesized in 93% yield: ¹H NMR (300 MHz, DMSO-d₆) δ5.38 (s, 4 H), 7.22 (d, J=8.9 Hz, 4 H), 7.41 (dd, J=5.5, 3.5 Hz, 2 H), 7.56 (dd, J=5.5, 3.5 Hz, 2 H), 7.89 (d, J=8.9 Hz, 4 H); ¹³C NMR (75 MHz, DMSO-d₆) δ68.5, 116.3, 125.8, 129.4, 129.8, 132.8, 135.4, 164.4, 167.3, 188.0; ESI (M−H⁺, negative ion mode) calcd for C₂₄H₁₇O₈ 433.0923, found 433.0921.

### Example 18

Ketoacid 5. This compound was synthesized in 91% yield: ¹H NMR (300 MHz, DMSO-d₆) δ5.26 (s, 4 H), 7.22 (d, J=8.8 Hz, 4 H), 7.50 (s, 4 H), 7.90 (d, J=8.8 Hz, 4 H); ¹³C NMR (75 MHz, DMSO-d₆) δ70.3, 115.5, 116.3, 125.7, 128.9, 132.9, 137.0, 164.5, 167.3, 188.0; ESI (M−H⁺, negative ion mode) calcd for C₂₄H₁₇O₈ 433.0923, found 433.0938.

### Example 19

Ketoacid 6. This compound was synthesized in 51% yield: ¹H NMR (300 MHz, DMSO-d₆) δ5.30 (s, 6 H), 7.22 (d, J=8.9 Hz, 6 H), 7.58 (s, 3 H), 7.90 (d, J=8.8 Hz, 6 H); ¹³C NMR (75 MHz, DMSO-d₆) δ70.3, 116.3, 125.8, 127.9, 132.8, 137.8, 164.4, 167.4, 188.1; ESI (M−H⁺, negative ion mode) calcd for C₃₃H₂₃O₁₂ 611.1190, found 611.1214.

Experimental Procedures for Enzyme Assays

### Example 20

Yersinia and LAR PTPase Assays: The phosphatase activities of the Yersinia and LAR PTPases (purchased from Calbiochem) were assayed using p-NPP as the substrate and the reaction progress was monitored by UV spectroscopy. Initial rates were determined by monitoring the hydrolysis of p-NPP at 420 nm, from 10 to 120 s after mixing. Assay solutions contained 2.5 mM substrate, 1 mM EDTA, 100 mM NaCl, 100 mM acetate at pH 5.5, and 10% DMSO. IC₅₀ values were calculated using a Dixon analysis. Data analysis was performed with the commercial graphing package Grafit (Erithacus Software Ltd.). The Kₘ values under these conditions were found to be 2.5 and 2.1 mM for Yersinia and LAR, respectively.

### Example21

PTP1B Assay: PTP1B (purchased from Calbiochem) was assayed usingp-NPP as the substrate and the reaction progress was monitored by UV spectroscopy. Initial rates were determined by monitoring the hydrolysis of p-NPP at 420 nm, from 10 to 120 s after mixing. Assay solutions contained 1.2 mM substrate, 1 mM EDTA, 100 mM NaCl, and 100 mM acetate at pH 5.5, and 10% DMSO. IC₅₀ values were calculated using a Dixon analysis. Data analysis was performed with the commercial graphing package Grafit (Erithacus Software Ltd.). The Kₘ value under these conditions was measured to be 0.62 mM. The Kₘ value in the absence of DMSO and in acetate buffer at pH 5.5 is reported to be 0.75 mM.¹

TABLE 1
Inhibition of the Yersinia PTPase by α-ketocarboxylic acids^{a}

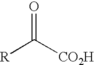

| No. | R | IC₅₀ (μM) |
|---|---|---|
| 1 | 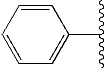 | 2,700 |
| 2 | 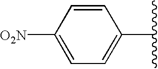 | 2,700 |
| 3 | 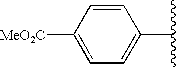 | 2,300 |
| 4 | 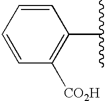 | 1,500 |
| 5 | 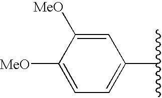 | 1,700 |
| 6 | 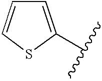 | 950 |
| 7 | 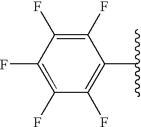 | 790 |
| 8 | 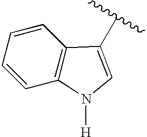 | 390 |
| 9 | 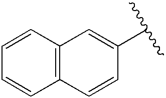 | 190 |
| 10 | 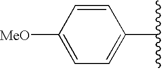 | 150 |
| 11 | 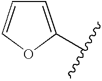 | 79^{b} |

^{a}Average of two measurements. The error in these values is approximately ±10%.
^{b}Kᵢ value.

TABLE 2
Inhibition of Phosphatases by α-Ketocarboxylic Acid-Based Inhibitors^{ab}

| | IC₅₀ (μM) | | |
|---|---|---|---|
| Compound | Yersinia PTPase | PTP1B | LAR |
| 3 | 8.5 ± 0.8 | 19 ± 5 | 120 ± 30 |
| 4 | 4.1 ± 0.6 | 53 ± 8 | 120 ± 15 |
| 5 | 0.7 ± 0.2 | 2.7 ± 0.5 | 250 ± 70 |
| 6 | 1.7 ± 0.5 | 13 ± 3 | 41 ± 5 |
| 27 | 200 ± 20 | 250 ± 30 | 540 ± 80 |

^{a}Average of two measurements.

Structures of compounds shown in Table 2.

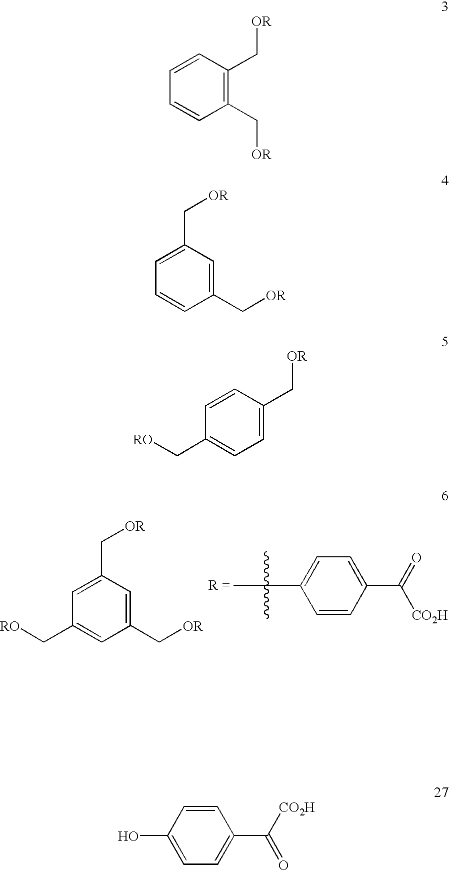

References

1. Iversen, L. F.; Andersen, H. S.; Branner, S.; Mortensen, S. B.; Peters, G. H.; Norris, K.; Olsen, O. H.; Jeppesen, C. B.; Lundt, B. F.; Ripka, W.; Moller, K. B.; Moller, N. P. H. Structure-based Design of a Low Molecular Weight, Nonphosphorus, Nonpeptide, and Highly Selective Inhibitor of Protein-tyrosine Phosphatase 1B. J. Biol. Chem. 2000, 275, 10300-10307.

2. Jerry March, Advanced Organic Chemistry, 4^{th} Edition, (publisher: J. Wiley & Sons), 1992.

All of the references and publications cited herein are hereby incorporated by reference.

Equivalents

Those skilled in the art will recognize, or be able to ascertain using no more than routine experimentation, numerous equivalents to the compounds and methods of use thereof described herein. Such equivalents are considered to be within the scope of this invention and are covered by the following claims.

We claim:

1. A compound having the general structure shown in Formula 1: 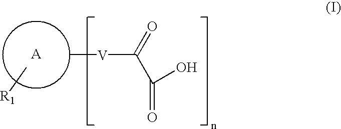
wherein, as valence and stability permits,
A represents an alkyl or heteroalkyl, or a substituted or unsubstituted aryl, heteroaryl, cycloalkyl, cycloalkenyl, heterocycloalkyl, or polycycle;
V, independently for each occurrence, is absent or represents a group selected from lower alkyl, alkoxy, arylalkyl, or a group with up to 10 heavy atoms;
R₁, independently for each occurrence, represents one or more of hydrogen, halogen, hydroxyl, alkoxyl, silyloxyl, amino, nitro, sulfhydryl, alkylthio, imine, amide, cyano, carbonyl, carboxyl, carboxamide, silyl, sulfamoyl, sulfinyl, thioalkyl, alkylsulfonyl, arylsulfonyl, ketone, aldehyde, ester, isocyano, guanidine, amidine, acetal, ketal, amine oxide, azide, carbamate, hydroxamic acid, imide, oxime, sulfonamide, thioamide, thiocarbonate, urea, thiourea, or a substituted or unsubstituted alkyl or heteroalkyl, alkenyl, alkynyl, aryl or heteroaryl or —(CH₂)ₘ-R₃;
R₃ represents a substituted or unsubstituted aryl, cycloalkyl, cycloalkenyl, heterocycloalkyl, heterocycle, or polycycle;
m, independently for each occurrence, represents an integer from 0 to 6; and
n is an integer from 1 to 4, or
a salt, an ester or amide derivative thereof.

2. The compound of claim 1 wherein, R₁, independently for each occurrence, represents one or more hydrogen, halogen, alkyl, alkoxy, cyano, amino, nitro or carbonyl group.

3. A compound having the general structure shown in Formula II: 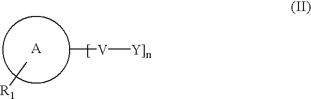
wherein, as valence and stability permits,
A represents an a substituted or unsubstituted aryl, heteroaryl, cycloalkyl, cycloalkenyl, heterocycloalkyl, or polycycle;
V, independently for each occurrence, is absent or represents a group selected from lower alkyl, alkoxy, arylalkyl, or: a group with up to 10 heavy atoms;
Y, independently for each occurrence, represents, 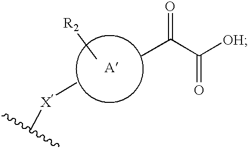
A′, independently for each occurrence, represents an alkyl or heteroalkyl, or a substituted or unsubstituted aryl, heteroaryl, cycloalkyl, cycloalkenyl, heterocycloalkyl, or polycycle;
X′, independently for each occurrence, represents an O, NR₅, or S;
R₁ and R₂, independently for each occurrence, represent one or more of hydrogen, halogen, hydroxyl, alkoxyl, silyloxyl, amino, nitro, sulfhydryl, alkylthio, imine, amide, cyano, carbonyl, carboxyl, carboxamide, silyl, sulfamoyl, sulfinyl, thioalkyl, alkylsulfonyl, arylsulfonyl, ketone, aldehyde, ester, isocyano, guanidine, amidine, acetal, ketal, amine oxide, azide, carbamate, hydroxamic acid, imide, oxime, sulfonamide, thioamide, thiocarbonate, urea, thiourea, or a substituted or unsubstituted alkyl or heteroalkyl, alkenyl, alkynyl, aryl or heteroaryl or —(CH₂)ₘ-R₃;
R₃ represents a substituted or unsubstituted aryl, cycloalkyl, cycloalkenyl, heterocycloalkyl, heterocycle, or polycycle;
R₅ represents a hydrogen or a group with up to 10 heavy atoms;
m, independently for each occurrence, represents an integer from 0 to 6; and
n is an integer from 1 to 4,
a salt, an ester or amide derivative thereof.

4. The compound of claim 3 wherein, R₁ and R₂, independently for each occurrence, represent one or more hydrogen, halogen, alkyl, alkoxy, cyano, amino, nitro or carbonyl group.

5. The compound of claim 1 or 3, wherein A represents a substituted or unsubstituted phenyl, naphthyl, furan, or thiofuran ring.

6. The compound of claim 1 or 3, wherein n is 2 or 3.

7. The compound of claim 1 or 3, wherein m is 1 or 2.

8. A pharmaceutical preparation comprising a sterile pharmaceutical excipient and a compound of claim 1 or 3.

9. The pharmaceutical preparation of claim 8, wherein the compound inhibits mammalian, bacterial, fungal or yeast protein tyrosine phosphatase.

10. The pharmaceutical preparation of claim 9, wherein the compound has a Kᵢ for inhibiting PTP activity of 25 μM or less.

11. The pharmaceutical preparation of claim 9, wherein the compound has a Kᵢ for inhibiting PTP activity of 10 μM or less.

12. The pharmaceutical preparation of claim 9, wherein the compound has a Kᵢ for inhibiting PTP activity of 1 μM or less.

13. The pharmaceutical preparation of claim 9, wherein the inhibitor has an IC₅₀ for inhibiting PTP activity of 10 μM or less.

14. The pharmaceutical preparation of claim 9, wherein the inhibitor has an IC₅₀ for inhibiting PTP activity of 1 μM or less.

15. A pharmaceutical dosage form comprising the preparation of claim 8.

16. The dosage form of claim 15, wherein said dosage form is a tablet or a capsule or oral solution.

17. The dosage form of claim 15, wherein said dosage form is adapted for intravenous infusion, parenteral delivery or oral delivery.

18. The dosage form of claim 15, wherein a therapeutically effective amount of the pharmaceutical dosage is in the range from about 0.1 mg to about 1000 mg.

19. The dosage form of claim 15, wherein said effective amount of the pharmaceutical dosage comprises and amount of the compound in the range from about 1 mg to about 500 mg.

20. The dosage form of claim 15, wherein said effective amount of the pharmaceutical dosage comprises and amount of the compound in the range of from about 10 mg to about 250 mg.

21. The dosage form of claim 15, wherein said effective amount of pharmaceutical dosage comprises and amount of the compound in the range of from about 10 mg to about 100 mg.

22. A method for synthesizing a compound with a general structure shown in Formula III, 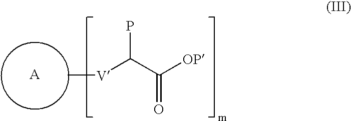
wherein, as valence and stability permits,
A represents an alkyl or heteroalkyl, or a substituted or unsubstituted aryl, heteroaryl, cycloalkyl, cycloalkenyl, heterocycloalkyl, or polycycle;
P represents a ketone protecting group or an optionally protected amine or hydroxyl;
P′ represents a protecting group for a carboxylic acid;
V′, independently for each occurrence, is absent or represents alkyl, alkenyl, alkynyl, alkoxy, heteroalkyl, or a substituted or unsubstituted aryl, heteroaryl, cycloalkyl, cycloalkenyl, heterocycloalkyl, or polycycle;
m, independently for each occurrence, represents an integer from 1 to 4;
comprising,
Reacting G with one or more equivalents of H in the presence of a base, wherein, as valence and stability permit,
G represents 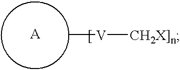
A represents alkyl or heteroalkyl, or a substituted or unsubstituted aryl, heteroaryl, cycloalkyl, cycloalkenyl, heterocycloalkyl, or polycycle;
V, independently for each occurrence, is absent or represents lower alkyl, alkenyl, alkynyl, alkoxy, or a group with up to 10 heavy atoms;
X is a leaving group preferably selected from halides, permethylated amines, acetates or sulphonate esters, such as a tosylate or mesylate; and
n represents a integer in the range of 1 to 4; and
H is represented by the general structure: 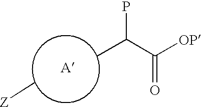
A′ represents an alkyl or heteroalkyl, or a substituted or unsubstituted aryl, heteroaryl, cycloalkyl, cycloalkenyl, heterocycloalkyl, or polycycle;
Z represents an OH, NR₅H, or SH;
R₅ represents a hydrogen or a group with up to 10 heavy atoms;
P′ represents a carboxyl protecting group; and
P represents a ketone protecting group or an optionally protected amine or hydroxyl.

23. A method for synthesizing a compound with a general structure shown in Formula IV, 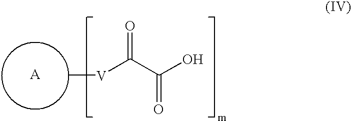
wherein, as valence and stability permits,
A represents an alkyl or heteroalkyl, or a substituted or unsubstituted aryl, heteroaryl, cycloalkyl, cycloalkenyl, heterocycloalkyl, or polycycle;
V, independently for each occurrence, is absent or represents a linking group selected from lower alkyl, alkenyl, alkynyl, alkoxy, or a group with up to 10 heavy atoms; and
m, independently for each occurrence, represents an integer from 1 to 4;
comprising the acts of
converting P to an oxo, and P′ to a hydrogen in the structure represented by formula IVa, 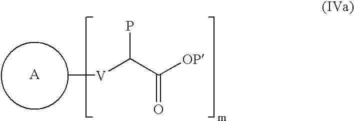
wherein, as valence and stability permits,
A represents an alkyl or heteroalkyl, or a substituted or unsubstituted aryl, heteroaryl, cycloalkyl, cycloalkenyl, heterocycloalkyl, or polycycle;
V, independently for each occurrence, is absent or represents a linking group selected from lower alkyl, alkenyl, alkynyl, alkoxy, or a group with up to 10 heavy atoms;
P represents a ketone protecting group or an optionally protected amine or hydroxyl;
P′ represents a protecting group for a carboxylic acid; and
m, independently for each occurrence, represents an integer from 1 to 4.

24. The method of claim 23, wherein P and P′ are converted in a substantially simultaneous manner.

25. The method of claim 23, wherein P and P′ are converted in a sequential manner.

26. A method for synthesizing a compound with a general structure shown in Formula V, 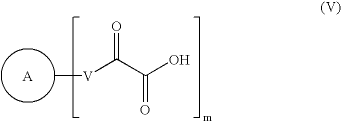
wherein, as valence and stability permits,
A represents an alkyl or heteroalkyl, or a substituted or unsubstituted aryl, heteroaryl, cycloalkyl, cycloalkenyl, heterocycloalkyl, or polycycle;
V, independently for each occurrence, is absent or represents a linking group selected from lower alkyl, alkenyl, alkynyl, alkoxy, or a group with up to 10 heavy atoms;
m, independently for each occurrence, represents an integer from 1 to 4;
comprising the act of converting P to an oxo in the compound represented by Formula Va: 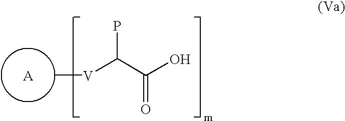
wherein, as valence and stability permits,
A represents an alkyl or heteroalkyl, or a substituted or unsubstituted aryl, heteroaryl, cycloalkyl, cycloalkenyl, heterocycloalkyl, or polycycle;
P represents a ketone protecting group;
V, independently for each occurrence, is absent or represents a linking group selected from lower alkyl, alkenyl, alkynyl, alkoxy, or a group with up to 10 heavy atoms; and
m, independently for each occurrence, represents an integer from 1 to 4.

27. A method for synthesizing a compound with a general structure shown in Formula VI, 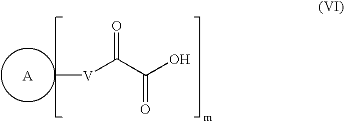
wherein, as valence and stability permits,
A represents an alkyl or heteroalkyl, or a substituted or unsubstituted aryl, heteroaryl, cycloalkyl, cycloalkenyl, heterocycloalkyl, or polycycle;
V, independently for each occurrence, is absent or represents a linking group selected from lower alkyl, alkenyl, alkynyl, alkoxy, or a group with up to 10 heavy atoms;
m, independently for each occurrence, represents an integer from 1 to 4;
comprising the act of converting P′ to a hydrogen in the compound represented by Formula VIa: 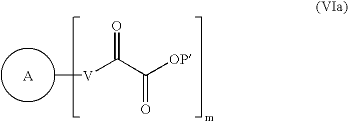
wherein, as valence and stability permits,
A represents an alkyl or heteroalkyl, or a substituted or unsubstituted aryl, heteroaryl, cycloalkyl, cycloalkenyl, heterocycloalkyl, or polycycle;
P′ represents a carboxy protecting group;
V, independently for each occurrence, is absent or represents a linking group selected from lower alkyl, alkenyl, alkynyl, alkoxy, or a group with up to 10 heavy atoms; and
m, independently for each occurrence, represents an integer from 1 to 4.

28. The method of claims 22, 23, 26 or 27 wherein, A represents a benzyl, phenyl, naphthyl, furan or thiofuran group.

29. The method of claims 22, 23, or 26 wherein, P represents a ketal or a tert-butoxycarbonyl protected amine.

30. A method for treating a PTP-mediated disorder, comprising administering to a subject diagnosed with a PTP-mediated disorder a therapeutically effective amount of compound of claim 1 or 3.

31. The method of claim 30, wherein the PTP-mediated disorder is selected from diabetes, obesity, autoimmune diseases, dysfunctions of the coagulation system, allergic diseases, diseases with decreased or increased synthesis or effects of growth hormones, osteoporosis, and proliferative disorders including cancer or psoriasis.

32. The method of claim 30, wherein the PTP-mediated disorder is selected from glucose intolerance, type I diabetes, type II diabetes, insulin resistance, and obesity.

33. A method for modulating glucose levels in a subject comprising administering a therapeutically effective amount of the compound of claims 1 or 3.

34. A method for using the compound of claim 1 or 3 as a diagnostic tool to elucidate a PTP-mediated metabolic pathway, wherein such pathway comprises signal transduction processes in which protein tyro sine phosphatase dephosphorylates a phosphorylated tyro sine residue.

35. A method for using the compound of claim 1 or 3 in a screening assay to identify PTP-mediated disease states.

36. A method for conducting a pharmaceutical business, comprising:
a. manufacturing a pharmaceutical preparation of claim 8; and
b. marketing to healthcare providers the benefits of using the composition in the treatment of a PTP-mediated disorder, disease or dysfunction.

37. A method for conducting a pharmaceutical business, comprising:
a. providing a distribution network for selling a pharmaceutical preparation of claim 8; and
b. providing instruction material to patients or physicians for using the preparation in the treatment of a PTP-mediated disorder, disease or dysfunction.

38. A method for conducting a pharmaceutical business, comprising:
a. determining an appropriate formulation and dosage of a pharmaceutical preparation of claim 8;
b. conducting therapeutic profiling of formulations identified in step (a), for efficacy and toxicity in animals; and
c. providing a distribution network for selling a preparation or preparations identified in step (b) as having an acceptable therapeutic profile.

39. The method of claim 38, including an additional step of providing a sales group for marketing the preparation to healthcare providers.

40. A method for conducting a pharmaceutical business, comprising:
a. determining an appropriate formulation and dosage of a pharmaceutical preparation of claim 8; and
b. licensing, to a third party, the rights for further development and sale of the formulation.
